(19) United States
(12) Patent Application Publication
(10) Pub. No.: US 2001/0005715 A1
(43) Pub. Date: Jun. 28, 2001

(54) Metal ion complexing agents

(76) Inventor: Yoram Tsivion, Givat Ada (IL)

Correspondence Address: Gifford, Krass, Groh, Sprinkle,, Anderson & Citkowski, P.C., Suite 400, 280 N. Old Woodward Ave., Birmingham, MI, 48009, US

(21) Appl. No.: 09/773,867

(22) Filed: Feb. 1, 2001

Related U.S. Application Data
(63) Continuation of application No. 09/174,714, filed on Oct. 19, 1998, now abandoned.
(60) Provisional application No. 60/063,606, filed on Oct. 20, 1997.

(51) Int. Cl. C07H 17/00; C07H 15/00; C07H 17/02; A61K 31/70; A61K 31/715

(52) U.S. Cl. 514/25; 536/4.1; 514/53; 536/6; 536/17.2; 536/18.2

(57) ABSTRACT

A metal ion complexing compound comprising a complexing agent formed of a condensation product of an aldose and a substituted benzene. The substituted benzene includes one or more groups comprising -OH, -NH₂, -COOH, or -SO₃H. The compound can be complexed with at least one metal ion and used for the administration of metal ions to plants and animals.

This application claims priority of United States Provisional Application Serial No. 60/063,606 filed Oct. 20, 1997.

### FIELD OF THE INVENTION

This invention relates generally to a field of fertilizers and concerns agents of which form complexes with metal ions microelements (hereafter “complexing agents”) for the purpose of administering these metal ions into roots of plants.

### BACKGROUND OF THE INVENTION

Various metal ions are essential elements in plant nutrition and soil deficiency thereof is very often the cause of poor plant growth and yield. Of particular importance for plant growth are ions of iron, manganese, copper and zinc, iron being especially important in calcareous soils. These metal ions tend to form highly insoluble complexes and this fact considerably limits their availability to plant roots.

Iron ions for example, are present in the soil either in their di-(Fe⁺²) or trivalent (Fe⁺³) form, neutral or alkaline conditions favoring the latter. In the trivalent form, iron ions form highly stable precipitates, having a solubility product with hydroxide in the order of 10⁻³⁸. Thus, in soils having a neutral or basic pH, such as calcarcous or sodic soils in which the high pH is buffered, iron precipitates rapidly and thus becomes unavailable to plants roots. Attempts were made to administer iron in a divalent ionic salt, but such administration proved to be not sufficiently efficient. Similar problems to iron, are also experienced in the administration of the other metal ions mentioned above.

Accordingly, in order to administer such metal ions to plants roots, it is necessary to use appropriate complexing agents. Such agents which were customarily used in fertilizers included EDDHA or EDDHMA. However, a major disadvantage of these complexing agents is their high price.

### SUMMARY OF THE INVENTION

It is an object of the present invention to provide novel complexing agents of metal ions microelements.

It is a further object of the present invention to provide novel fertilizer compositions comprising the novel complexing agent and the above metal ions.

It is a further object of the present invention to provide a novel method for administering metal ions to plant roots.

It is a still further object of the present invention to provide a process for preparing a complexing agent for the above metal ions.

### DETAILED DESCRIPTION OF THE PREFERRED EMBODIMENTS

In accordance with the present invention, novel condensation products of certain benzene derivatives with aldoses have been found to possess highly beneficial properties as complexing agents of metal ion microelements.

The present invention thus provides a condensation product of an aldose and benzene derivatives substituted by one or more members selected from the group consisting of -OH, -NH₂ and -COOH and optionally also by -SO₃H.

Examples of said benzene derivatives are hydroxybenzoic acid, phenol, aminophenol, p-cresol, aminophenolsulfonic acid, dihydroxysulfonic acid, soluble lignin derivatives, tannic acid and water soluble tannic acid derivatives. Particular examples are salicylic acid; 2- or 3-aminophenol; dihydroxysulfonylbenzoic acid; coniferyl alcohol, coumaric acid.

Examples of aldoses are monohexoses such as glucose, mannose, galactose or reducing dihexoses such as lactose.

The condensation reaction is preferably performed at high, i.e, alkalinic pH.

It has been further found in accordance with the invention that certain phenols, hydroxybenzoic acid and aminobenzene derivatives carrying one or more sulfonyl groups as a ring substituent also possess highly beneficial properties as complexing agents of metal ion microelements.

Examples of these phenolsulfonyl derivatives are sulfonated dihydroxyphenols, sulfonated ,hydroxybenzoic acids, sulfonated phenols, sulphonated aminophenols, dihydroxydisulfonilbenzoic acids, such as Tiron™; dihydroxybenzensulfonilic acid such as 1,2- or 1,3-dihydroxybenesulfonic acids.

The present invention also provides agricultural preparation useful both as a fertilizer of plants and as a nutritional solution for livestock comprising metal ion microelements and a complexing agent of said microelements, the complexing agent being a member selected from the group consisting of the condensation products and the sulfonyl derivatives.

The composition of the present invention can be an aqueous liquid in a form ready for use as a fertilizer, be a concentrate for dilution with an aqueous solution prior to administration or may be in a dry e.g., powdered form.

By another of its aspects, the present invention comprises a method for administering metal ions to plants roots, comprising administering said metal ions together with a complexing agent comprising such condensation product.

The invention will now be illustrated with reference to the following examples.

### EXAMPLE 1

1 gr of pyrocatechol and 2 gr of glucose were dissolved in 30 ml water. 2 ml of 20% (w/v) NaOH solution was added and the mixture was then stirred and heated to about 70° C. for a period of 1-2 hours.

To the resulting dark brown solution, 2.5 g FeSO₄·7H₂O were added (ca. 1:1 molar ratio iron with pyrocatechol) and then fresh water was added under continuous stirring until the iron salt was completely dissolved. The resulting solution was then dried by heating it to about 70° C. A dark-purple colored powder weighing about 4-5 gr was obtained.

It should be noted that the amount of glucose reacted may be varied between 2-6 gr and the amount of NaOH solution added can be varied between 1-3 ml without considerable effect on the resulting product.

### EXAMPLE 2

Petunia plants were grown in winter time in calcareous soil until they were completely chlorotic evidenced by the very pale green color of their leaves. 2 gr of the powdered substance obtained in Example 1 was administered in dry form to the soil around the plants and the ground was watered several times to enhance dissolution of the preparation. Greening of the plants started between 4 to 6 days after administration and the plants continued to gain a green color until, after a few weeks, the color of their leaves turned to the normal dark green.

Untreated plants which served as controls, withered and most of them died within this period.

### EXAMPLE 3

Soybean seeds of the Williams variety were sown in May in calcareous soil. Plants were fertilized with a standard NPK fertilizer two weeks after germination. Plants gradually turned chlorotic evidenced by the fact that the production of new leaves stopped and old leaves slowly became yellow and withered. 1 gr of the powder obtained in Example 1 was administered around the stem of each randomly chosen plant, and the soil was then watered several times to enhance dissolution of the substance. Untreated plants served as control and were irrigated similarly as the treated plants.

After four days top leaf veins began to turn green and in the course of the next few days, they turned completely dark green, with plants resuming leaf production. Untreated controlled pl ants di d not turn green and did not resume growth .

### EXAMPLE 4

Sulphonated catechol

1 gr of catechol was mixed with 1 ml of water and to this mixture 3 ml of concentrated sulfuric acid was added. The mixture was then heated to about 70° C. for ten minutes until a brownish solution of sulfonated catechol was obtained. To this solution, about 2.5 gr of FeSO₄·7H₂O and 20 ml of 20% (w/v) NaOH solution were added and the resulting solution was stirred and then dried at a temperature of about 70° C.

Plants were treated with the resulting dried powder similarly as in Example 2, and similar results, ie., curing plants from chlorosis, were obtained.

### EXAMPLE 5

Sulfonyl catechol prepared as Example 4 was mixed with 1.5 gr SO₄ MnSO₄·1H₂O, neutralized with NaOH and dried in the oven at a temperature of about 70° C. 1 gr of the dried powder was administered to two parts of highly chlorotic and retarded rose plants, which were previously unsuccessfully treated with iron and zinc fertilizers. The plants turned green within a few weeks and resumed growth.

### EXAMPLE 6

Resorcinol was condensed with glucose by similarity as in the case of catechol in Example 1. The condensation product was mixed with 1.5 g of ZnCl₂.

The resulting method complex was shown to be very stable in water saturated with calcium carbonate for several weeks, evidenced by the fact that the solution remained clear and no precipitate was found. Preliminary results performed with rose plants, which suffered from zinc deficiencies showed an improvement in the plants' condition following application of the above solution, evidenced by resumption of growth.

In view of the teaching presented herein, other modifications and variations of the present inventions will be readily apparent to those of skill in the art. The foregoing drawings, discussion, and description are illustrative of some embodiments of the present invention, but are not meant to be limitations on the practice thereof It is the following claims, including all equivalents, which define the scope of the invention.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to, carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed:

1. A metal ion complexing compound, said compound comprising:
a complexing agent comprising a condensation product of an aldose and a substituted benzene, said substituted benzene including one or more groups comprising -OH, -NH₂, -COOH, or -SO₃H.

2. A compound according to claim 1, wherein said complexing agent is a condensation product of said aldose and a compound selected from the group consisting of hydroxybenzoic acid, amino phenol, dihydroxysulfonic acid, soluble lignin derivatives, tannic acid and water soluble tannic acid derivatives.

3. A compound according to claim 1, wherein said complexing agent is a condensation product of said aldose and a group selected from the group consisting of salicylic acid, 2- or 3-aminophenol, dihydroxysulfonilbenzoic acid, p-coumaryl, sinapyl alcohol, tannic acid and water soluble tannic acid derivatives.

4. A compound according to claim 1, wherein said aldose is a mono- or reducing di-hexose.

5. A compound according to claim 1, wherein said complexing agent is a phenol derivative carrying a sulfonic group as a ring substituent, said benzene derivative being selected from the group consisting of water soluble dihydroxybenezenes, hydroxybenzoic acids, phenols, aminophenols, aminobenezenes, water soluble lignin derivatives and tannic acid, which derivatives carrying one or more sulfones.

6. A metal ion complexing compound, said compound comprising:
a water soluble phenol, phenolcarboxylic acid and amino-benzene derivatives having at least one sulphonic group as a ring substituent.

7. A method of delivering metal ions to plants and animals, said method comprising:
administering an effective amount of at least one metal ion complexed with a complexing agent comprising a condensation product of an aldose and a substituted benzene, the benzene being substituted with -OH, -NH₂, -COOH, or -SO₃H.

8. A method according to claim 7, wherein said complexing agent is a condensation product of the aldose and a compound selected from the group consisting of hydroxybenzoic acid, amino phenol, dihydroxysulfonic acid, soluble lignin derivatives, tannic acid and water soluble tannic acid derivatives.

9. A method according to claim 7, wherein the complexing agent is a condensation product of the aldose and a group selected from the group consisting of salicylic acid, 2- or 3-aminophenol, dihydroxysulfonilbenzoic acid, p-coumaryl, sinapyl alcohol, tannic acid and water soluble tannic acid derivatives.

10. A method according to claim 7, wherein the aldose is a mono- or reducing di-hexose.

11. A method according to claim 7, wherein the complexing agent is a phenol derivative carrying a sulfonic group as a ring substituent, the benzene derivative being selected from the group consisting of water soluble dihydroxybenezenes, hydroxybenzoic acids, phenols, aminophenols, aminobenezenes, water soluble lignin derivatives and tannic acid, which derivatives carrying one or more sulfones.

12. A method for delivering metal ions to plants and animals, said method comprising:
administering an effective amount of at least one metal ion complexed with a complexing agent comprising a water soluble phenol, phenolcarboxylic acid and amino-benzene derivatives having at least one sulphonic group as a ring substituent.
